(19) United States
(12) Patent Application Publication
(10) Pub. No.: US 2007/0122417 A1
(43) Pub. Date: May 31, 2007

(54) Immunotherapy method

(76) Inventors: Patrick Holt, Nedlands (AU); Wayne Thomas, Nedlands (AU); Titiek I. Tobagus, Jakarta (ID)

Correspondence Address: EDWARDS ANGELL PALMER & DODGE LLP, P.O. BOX 55874, BOSTON, MA, 02205, US

(21) Appl. No.: 10/574,393

(22) PCT Filed: Sep. 29, 2004
(86) PCT No.: PCT/AU04/01333

§ 371 Date: Jan. 12, 2007

(30) Foreign Application Priority Data
Sep. 30, 2003 (AU) 2003905314

(51) Int. Cl. A61K 39/00

(52) U.S. Cl. 424/184.1

(57) ABSTRACT

The present invention relates to the use of immunomodifying agents to effect change in the T helper-type 1 (TH1) or T helper-type 2 (TH2) arms of the immune response and thereby treat TH1 or TH2 mediated diseases. In particular, the present invention relates to a method of altering a specific immune response in an individual comprising: i). administering to an individual in need thereof an effective amount of an antigen in immunotherapeutic form, wherein said immune response is down regulated; and ii). subsequently administering to the individual an effective amount of an immunomodifying agent comprising said antigen in immunogenic form.

### FIELD OF THE INVENTION

The present invention relates to the use of immunomodifying agents to effect change in the T helper-type 1 (TH1) or T helper-type 2 (TH2) arms of the immune response and thereby treat TH1 or TH2 mediated diseases. In particular, the present invention relates to the use of immunomodifying agents comprising specific antigen(s) alone or together with adjuvant(s) to effect change in the TH1 or TH2 immune responses.

### BACKGROUND OF THE INVENTION

Strongly polarized TH1 and TH2 responses not only play different roles in protection, but can also promote different immunopathological reactions. Indeed, many diseases are thought to involve a pathologic or inappropriate immune response either by the TH1 branch of the immune response which is associated primarily with cell mediated immunity, or by the TH2 branch which primarily drives antibody production. The interplay and importance of various aspects of the immune response, including interaction between TH1 and TH2 cell cytokines is discussed in WO97/26883. Although W097/26883 is specifically concerned with the effects of a particular antiviral compound known as Ribavirin®, it nonetheless illustrates some of the complex and unpredictable effects of drug compounds on the immune system.

The TH2 branch of the immune system is generally directed at protecting against extracellular pathogens such as parasites through the production of antibodies by B cells in particular IgE; whereas the TH1 branch is generally directed at intracellular pathogens such as viruses through the activity of natural killer cells, cytotoxic T lymphocytes and activated macrophages, and the cytokines secreted by these cells. TH2 cells are believed to produce cytokines which include IL-3, IL-4, IL-5, and IL-13, which are thought to stimulate production of IgE antibodies, as well as be involved with recruitment, proliferation, differentiation, maintenance and survival of eosinophils (ie., leukocytes that accept an eosin stain) and regulation of the functions of other cell types.

It is generally known that TH1 and TH2 responses are controlled by “cross regulation”. For example, TH1 cytokines can actively inhibit the growth and differentiation of TH2 cells and vice versa (See, for example, Zhang, 2001, J. Ex. Med. 194:165-172; Murphy, 1996, J. Ex. Med. 183: 901-913; O'Garra, 1998, Immunity. 8:275-283).

Uncontrolled TH1 type responses are involved in organ specific autoimmunity such as rheumatoid arthritis, multiple sclerosis, thyroiditis, Crohn's disease, systemic lupus erythematosus, experimental autoimmune uveoretinitis (Dubey et al., 1991, Eur. Cytokine Network, 2:147-152), experimental autoimmune encephalitis (EAE) (Beraud et al., 1991, Cell Immunol. 133:379-389) and insulin dependent diabetes mellitus (Hahn et al., 1987, Eur. J. Immunol. 18:2037-2042), in contact dermatitis (Kapsenberg et al., Immunol Today, 12:392-395), and in some chronic inflammatory disorders. The principal inflammatory cytokine produced by TH1 cells is IFN γ (See, for example, Romragnani, ed, TH1 and TH2 Cells in Health and Disease. Chem. Immunol., Karger, Basel, 63, pp. 158-170 and 187-203 (1996)).

In contrast, uncontrolled TH2 type responses are responsible for triggering allergic atopic disorders (against common environmental allergens) such as allergic asthma (Walker et al., 1992, Am. Rev. Resp. Dis. 148:109-115) and atopic dermatitis (van der Heijden et al., 1991, J. Invest. Derm. 97:389-394). TH2 type responses are also preferentially induced in certain primary immunodeficiencies such as hyper-IgE syndrome (Del Prete et al., 1989, J. Clin. Invest. 84:1830-1835) and Omenn's syndrome (Schandene et al., 1993, Eur. J. Immunol. 23:56-60). Other conditions associated with excessive TH2 type response are eczema, psoriasis, allergic rhinitis and hay fever (See, for example, Romragnani, supra).

Thus, it is clear that modulation of TH1 or TH2 responses involved in the aforementioned disease states would be of therapeutic benefit. In particular it would be of major benefit if it was possible to simultaneously modulate both the intensity of a specific disease-associated immune response, while at the same time controlling the TH1/TH2 balance within that immune response.

With the foregoing in mind, the inventors have now surprisingly found methods that selectively attenuate a host's antigen-specific TH1 or TH2 response thereby alleviating or overcoming TH1 or TH2 associated disease conditions.

### SUMMARY OF THE INVENTION

Accordingly, in one aspect, the present invention provides a method of altering a specific immune response in an individual comprising:
i). administering to an individual in need thereof an effective amount of an antigen in immunotherapeutic form, wherein said immune response is down regulated; and
ii). subsequently administering to the individual an effective amount of an immunomodifying agent comprising said antigen in immunogenic form.

Preferably, the immunomodifying agent further comprises either a TH1 or TH2 adjuvant, wherein the adjuvant normally induces the type of TH-response which is the target of the immunotherapy.

Preferably, the immunotherapy is targeted at the specific immune response.

In one embodiment, the effective amount of antigen in immunotherapeutic form comprises one or more doses of the antigen. In another embodiment, the effective amount of antigen in immunotherapeutic form further comprises agents designed to modulate the specific immune responses.

Preferably, the alteration to the specific immune response is attenuation of the TH-response component, which is associated with expression of the disease being treated.

In one embodiment, the alteration to the specific immune response is conversion of the TH1 component of the response to a TH2 component or conversion of the TH2 component to a TH1 component.

In another embodiment, the alteration to the specific immune response is reversing the ratio between the TH1 and TH2 components of the response, such that an immune response in an untreated patient which comprised high level production of TH1 cytokines and low level production of TH2 cytokines was converted to an immune response comprising high level production of TH2 cytokines and low level production of TH1 cytokines, or vice versa.

In a second aspect, the present invention provides a method of treating a TH1-associated disease comprising:
i). administering to an individual in need thereof an effective amount of an antigen in immunotherapeutic form; and
ii). subsequently administering to the individual an effective amount of an immunomodifying agent comprising said antigen in immunogenic form, wherein the antigen specific TH1 response in the individual is reduced relative to the specific TH1 response before administration of said immunomodifying agent. Preferably, the immunomodifying agent further comprises a TH1 adjuvant.

In a third aspect, the present invention provides a method of treating a TH2-associated disease comprising:
i). administering to an individual in need thereof an effective amount of an antigen in immunotherapeutic form; and
ii). subsequently administering to the individual an effective amount of an immunomodifying agent comprising said antigen in immunogenic form, wherein the antigen specific TH2 response in the individual is reduced relative to the specific TH2 response before administration of said immunomodifying agent.

Preferably, the immunomodifying agent further comprises a TH2 adjuvant.

In a fourth aspect, the present invention provides a method of treating a disease associated with a mixed TH1 and TH2 immune response comprising:
i). administering to an individual in need thereof an effective amount of an antigen in immunotherapeutic form; and
ii). subsequently administering to the individual an effective amount of an immunomodifying agent comprising said antigen in immunogenic form which boosts both TH1 and TH2 immunity, wherein ensuing specific TH1 and TH2 responses in the individual are reduced relative to the specific TH1 and TH2 responses before administration of said immunomodifying agent.

Preferably, the immunomodifying agent further comprises either an adjuvant which boosts both TH1 and TH2 immunity or in a mixture of TH1 and TH2 adjuvants, wherein ensuing specific TH1 and TH2 responses in the individual are reduced relative to the specific TH1 and TH2 responses before administration of said immunomodifying agent.

In another embodiment the immunotherapy is administration of an effective amount of one or more antigen(s) in immunotherapeutic form, which antigens are associated with expression of pathogenic TH2 immunity to an individual in need thereof. In particular, if the disease is a TH1-associated disease then the antigen will predominately be a TH1-specific antigen.

In a fifth aspect, the present invention provides a method of treating a disease comprising:
i). administering to an individual in need thereof an effective amount of an antigen in immunotherapeutic form, wherein the immune response to said disease is down regulated; and
ii). subsequently administering to the individual an effective amount of an immunomodifying agent comprising said antigen in immunomodifying form.

Preferably, the immunomodifying agent further comprises either a TH1 or TH2 adjuvant, wherein the adjuvant normally induces the type of TH-response which is the target of the immunotherapeutic form of the antigen.

In one embodiment the disease is a TH1-associated disease. In particular, the TH1-associated disease is selected from the group consisting of rheumatoid arthritis, multiple sclerosis, thyroiditis, Crohn's disease, systemic lupus erythematosus, experimental autoimmune uveoretinitis, experimental autoimmune encephalitis, insulin dependent diabetes mellitus, contact dermatitis and chronic inflammatory disorders.

In another embodiment the disease is a TH2-associated disease. In particular, the TH2-associated disease is selected from the group consisting of allergic atopic disorders, allergic asthma, atopic dermatitis, hyper-IgE syndrome, Omenn's syndrome, and allergic rhinitis.

The TH1 or TH2 adjuvant may be any known adjuvant, which is specific for either TH1 or TH2 response, respectively. For example, TH2 adjuvants may be selected from the group consisting of alum, pertussis toxin, lacto fucopentaose III, and phosphopolymer or combinations thereof.

Preferred adjuvants for use in eliciting a predominantly TH1-type response may be selected from the group consisting of complete Freund's adjuvant, monophosphoryl lipid A, 3-de-o-acylated monophosphoryl lipid A (3D-MPL), aluminum salt, CpG-containing oligonucleotides, immunostimulatory DNA sequences, saponin, Montanide ISA 720, SAF, ISCOMS, MF-59, SBAS-3, SBAS-4, Detox, RC-529, aminoalkyl glucosaminide 4-phosphate, and LbeIF4A.

In one embodiment, the individual is a mammalian animal such as a dog, a cat, a livestock animal, a primate or a horse as well as a human. Preferably, the individual is a human subject.

In a sixth aspect, the present invention provides a kit for altering TH1 or TH2 response phenotype in an individual in need thereof comprising:
i). one or more TH1 antigen(s); or
ii). one or more TH1 or TH2 adjuvant(s); or
iii).combinations thereof; and
iv). instructions for use.

In an seventh aspect, the present invention provides a method of immunotherapy comprising:
i). administration to an individual in need thereof a plurality of antigen shots;
ii). administration to said individual less than five individual shots of said antigen combined with a TH1 and/or TH2 adjuvant.

Preferably, the individual shots of said antigen combined with a TH1 and/or TH2 adjuvant is less than three. More preferably, the number of individual shots of said antigen combined with a TH1 and/or TH2 adjuvant is one.

In an eighth aspect, the present invention provides a use of an immunomodifying agent for the manufacture of a medicament for the treatment a TH1-associated disease or TH2-associated disease, wherein said immunomodifying agent comprises an antigen in immunomodifying form.

Preferably, the immunomodifying agent further comprises at least one adjuvant that is associated with augmenting a T helper-response of the type associated with said disease.

Accordingly, in a ninth aspect, the present invention provides use of immunomodifying agent for the manufacture of a medicament for the treatment of a TH-1 or TH-2 associated disease inflicting an individual susceptible hereto, where said individual previously is treated with an immunotherapeutic form and dose of an antigen having reduced the T-helper immune response associated with said disease in said individual, and wherein the immunomodifying agent comprises at least one adjuvant that is associated with augmenting a T helper-response of the type associated with said disease and a immunogenic form of said antigen.

In a tenth aspect, the present invention provides an immunomodifying agent comprising at least one antigen in immunogenic form and at least one adjuvant, wherein the adjuvant normally induces the type of TH-response associated with the disease caused by said antigen.

The foregoing and other aspects of the present invention are explained in greater detail in the specification below.

### BRIEF DESCRIPTION OF THE FIGURES

FIG. 1 shows the selective tolerisation of TH2 immunity.

FIG. 2 shows selective tolerisation of TH1 immunity.

FIG. 3 shows non-selective tolerisation of overall OVA-specific TH-cell immunity.

FIG. 4 shows the desensitisation of OVA-sensitised mice.

FIG. 5 shows IgE in control mice treated with immunotherapeutic (sublingual) OVA but without the modifying immunogenic injection.

FIG. 6 shows IgE in mice treated with immunotherapeutic (sublingual) OVA with a mixed modifying immunogenic injection of OVA ip after the immunotherapy.

FIG. 7 shows IgE in mice treated with immunotherapeutic (sublingual) OVA with a Th2 modifying immunogenic injection of OVA in alum after the immunotherapy.

### DETAILED DESCRIPTION OF THE INVENTION

Before describing the present invention in detail, it is to be understood that this invention is not limited to particularly exemplified immunomodifying agents, antigens, adjuvants or methods and may, of course, vary. It is also to be understood that the terminology used herein is for the purpose of describing particular embodiments of the invention only, and is not intended to be limiting which will be limited only by the appended claims.

All publications, patents and patent applications cited herein, whether supra or infra, are hereby incorporated by reference in their entirety. However, publications mentioned herein are cited for the purpose of describing and disclosing the protocols, reagents and vectors which are reported in the publications and which might be used in connection with the invention. Nothing herein is to be construed as an admission that the invention is not entitled to antedate such disclosure by virtue of prior invention.

Furthermore, the practice of the present invention employs, unless otherwise indicated, conventional immunological techniques, chemistry and pharmacology within the skill of the art. Such techniques are well known to the skilled worker, and are explained fully in the literature. See, eg., Coligan, Dunn, Ploegh, Speicher and Wingfield “Current protocols in Protein Science” (1999) Volume I and II (John Wiley & Sons Inc.); and Bailey, J. E. and Ollis, D. F., Biochemical Engineering Fundamentals, McGraw-Hill Book Company, NY, 1986.

It must be noted that as used herein and in the appended claims, the singular forms “a,” “an,” and “the” include plural reference unless the context clearly dictates otherwise. Thus, for example, a reference to “a protein” includes a plurality of such proteins, and a reference to “an adjuvant” is a reference to one or more adjuvants, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

The present invention relates to methods of effecting, altering or enhancing a specific immune response in an individual. The term “specific immune response” as used herein refers to subjects' or individuals' response to a particular challenge ie whether the individual has a predominantly TH1 cell or predominantly TH2 cell response when challenged with a particular antigen. The terms “preferentially”, predominantly”, “substantially” and the like, when referring to TH1 or TH2 cells, mean that the cytokines produced by one particular TH cell type are more prevalent than the cytokines produced by the other TH cell type. For example, the term “predominantly TH1 cells” or an equivalent phrase means that the cytokines produced by TH1 cells eg IFN-γ, are more prevalent in an individual than TH2 cytokines eg IL-3, IL-4, IL-5, and IL-13.

As used herein with reference to the specific immune response the term “enhance”, or “enhanced” denotes a change in the total amount of one or more cytokines associated with a particular TH cell type. For example, the term “enhanced TH1 cells” or an equivalent phrase means that the cytokines produced by TH1 cells eg IFN-γ, are more prevalent than is normally present or IFN-γ is more prevalent than any of the TH2-associated cytokines. This may be evidenced by, for example, an observed increase in the amount of TH1-associated cytokines relative to TH2-associated cytokines. Or an increase in the amount of a TH1-associated cytokine relative to the amount of TH2-associated cytokine normally present.

The terms “altering or altered,” “effecting or effected” or “altering relative to” are all used herein to imply or suggest that the specific immune response of an individual has been modified when compared to specific immune response before the methods of the invention have been used. For example, if an individual has predominantly TH1-associated cytokines present before the methods disclosed herein are applied and upon application of the methods the TH2-associated cytokines become predominate, or at least closely approximating the levels of TH1-associated cytokines, then the TH1 cells would have been “altered” or “effected” by the methods of the invention “relative” to the TH2 cells.

The terms “subject” or “individual” are used interchangeably herein to refer to any member of the subphylum cordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The terms do not denote a particular age. Thus, both adult and newborn individuals are intended to be covered. The methods described herein are intended for use in any of the above vertebrate species, since the immune systems of all of these vertebrates operate similarly.

Thus, provided is the treatment of mammals such as humans, as well as those mammals of economical importance and/or social importance to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, eg., poultry, such as turkeys, chickens, ducks, geese, guinea fowl, and the like, as they are also of economical importance to humans. Thus, provided is the treatment of livestock, including, but not limited to, domesticated swine (pigs and hogs), ruminants, horses, poultry, and the like.

In one embodiment the individual is afflicted with a TH1-or TH2-associated disease. The term “TH1-associated disease” as used herein refers to a disease, which is mediated by TH1 cells or is associated with elevated levels of antigen-specific cytokine production, which in turn is associated with TH1 cells relative to the levels of TH2-associated cytokines. Such diseases include, but are not limited to organ specific autoimmunity such as rheumatoid arthritis, multiple sclerosis, thyroiditis, Crohn's disease, systemic lupus erythematosus, experimental autoimmune uveoretinitis (Dubey et al., 1991, Eur. Cytokine Network 2:147-152), experimental autoimmune encephalitis (EAE) (Beraud et al., 1991, Cell Immunol. 133:379-389) and insulin dependent diabetes mellitus (Hahn et al., 1987, Eur. J. Immunol. 18:2037-2042), in contact dermatitis (Kapsenberg et al., Immunol Today 12:392-395), and in some chronic inflammatory disorders.

The term “TH2-associated disease” as used herein refers to a disease, which is mediated by TH2 cells or is associated with elevated antigen-induced production of TH2 cytokines relative to TH1 cytokines. Such diseases include, but are not limited to TH2 type responses are responsible for triggering allergic atopic disorders (against common environmental allergens) such as allergic asthma (Walker et al., 1992, Am. Rev. Resp. Dis. 148:109-115) and atopic dermatitis (van der Heijden et al., 1991, J. Invest. Derm. 97:389-394).

Individuals with a TH1- or TH2-associated disease usually have elevated levels of TH1 or TH2 cytokine production, respectively. In “treating” these individuals with the methods disclosed herein the initial step involves either the individual “undergoing immunotherapy” or having “recently undergone immunotherapy” wherein the immunotherapy at least comprises the “administration” of one or more doses of an “effective amount” of a TH1 or TH2 antigen in a “immunotherapeutic form” to the individual or subject.

Generally, the terms “treating,” “treatment” and the like are used herein to mean affecting an individual or subject, their tissue or cells to obtain a desired pharmacological and/or physiological effect. The effect may be prophylactic in terms of completely or partially preventing the TH1- or TH2-associated disease or sign or symptom thereof, and/or may be therapeutic in terms of a partial or complete cure of TH1- or TH2-associated disease. “Treating” as used herein covers any treatment of, or prevention of TH1- or TH2-associated disease in a vertebrate, a mammal, particularly a human, and includes: (a) preventing the TH1- or TH2-associated disease from occurring in a subject that may be predisposed to the TH1- or TH2-associated disease, but has not yet been diagnosed as having them; (b) inhibiting the TH1- or TH2-associated disease, ie., arresting its development; or (c) relieving or ameliorating the symptoms of the TH1- or TH2-associated disease, ie., cause regression of the symptoms of the TH1- or TH2-associated disease.

The term “undergoing immunotherapy” means that the individual is receiving therapy for a disease or condition, which is designed to overcome or alleviate the symptoms of the disease or condition. In particular, the immunotherapy is administration of an antigen associated with the disease or condition in order to tolerise or downregulate the specific immune response of the individual. However, it will be appreciated that other immunotherapeutics may also be administered together with, prior to or subsequent to the antigen.

In one embodiment, the immunotherapy is the administration of a “immunotherapeutic form” of the antigen. A “immunotherapeutic form” of an antigen is a form of or formulation comprising the antigen, which down regulates (desensitises) the immune response to the antigen over time.

Several immunotherapeutic forms have already been proposed. (See, for example, U.S. Pat. No. 6,488,937 to Smits; U.S. Pat. No. 5,244,663 to Bruttmann et al.; GB-A-2 099 698 to Melillo; EP-A-0 135 022 to Moran; Glenis et al, Clinical Allergy, 1986, Vol. 16, 483-491; Mailing, H. J., (ed.), Immunotherapy Position Paper, Allergy (Supp.) 6, 43:9-33 (1988) all of which are incorporated in their entirety herein by reference.)

The immunotherapeutic form typically involves injecting into the individual gradually increasing doses of the antigen, usually to maximum tolerated doses (doses not giving rise to major allergic response), at varying intervals in an attempt to develop IgG antibody protection against the antigen, and to increase the specific suppressor T-Lymphocyte activity responding to antigen hypersensitivity.

The concentration and amount of the antigen in the immunotherapeutic form is dependent upon many factors, which are specific to the individual having the antigen hypersensitivity. It is therefore necessary to titrate the subject to determine the proper dosage. A variety of standard techniques are available to carry out this procedure, which are all well known in the art.

The term “recently undergone immunotherapy” refers to the same type of immunotherapy as described above, but also refers to the timing of any subsequent treatment. For example, the methods of the invention are best administered to an individual that is still under the effects of immunotherapy. Consequently, the term “recently” refers to a time point when the effects of the immunotherapy are still present.

The term “effective amount” of a TH1 or TH2 antigen means that the TH1 or TH2 antigen is sufficient to produce an effect on the TH1 or TH2 specific immune response. For example, in one embodiment the antigen is a TH1 specific antigen, which when administered in an “effective amount” would downregulate the specific immune response. The term “effective amount” when used with reference to the immunotherapeutic form encompasses one or more doses of a particular antigen.

An “antigen” is a substance that is recognised and bound specifically by an antibody or by a T cell antigen receptor. Antigens can include peptides, proteins, glycoproteins and polysaccharides, including portions thereof and combinations thereof. The antigens can be those found in nature or can be synthetic. The term “antigen” can also refer to any immunogenic moiety or agent, generally a macromolecule, which can elicit an immunological response in an individual. The term may be used to refer to an individual macromolecule or to a homogeneous or heterogeneous population of antigenic macromolecules. As used herein, “antigen” is generally used to refer to a hapten, an organic or inorganic substance, or a protein molecule or portion thereof which contains one or more epitopes. For purposes of the present invention, antigens can be obtained or derived from any known virus, bacteria, parasite or fungal pathogen, a plant, or from man-made or naturally occurring inorganic or organic material. The term also intends any of the various tumour-specific antigens and antigens associated with autoimmune diseases. Furthermore, for purposes of the present invention, an “antigen” includes a protein having modifications, such as deletions, additions and substitutions (generally conservative in nature) to the native sequence, so long as the protein maintains sufficient immunogenicity. These modifications may be deliberate, for example through site-directed mutagenesis, or may be accidental, such as through mutations of hosts which produce the antigens.

In various aspects of the invention, the antigen contains one or more T cell epitopes. A “T cell epitope” refers generally to those features of a peptide structure which are capable of inducing a T cell response. In this regard, it is accepted in the art that T cell epitopes comprise linear peptide determinants that assume extended conformations within the peptide-binding cleft of MHC molecules, (Unanue et al., 1987, Science, 236:551-557). As used herein, a T cell epitope is generally a peptide having at least about 7 amino acid residues, and preferably at least 8-18 or more amino acid residues. The ability of a particular antigen to stimulate a cell-mediated immunological response may be determined by a number of well-known assays, such as by lymphoproliferation (lymphocyte activation) assays, CTL cytotoxic cell assays, or by assaying for T-lymphocytes specific for the antigen in a sensitised subject. See, eg., Erickson et al., 1993, J. Immunol. 151:4189-4199; and Doe et al. (1994) Eur. J. Immunol. 24:2369-2376

In other aspects of the invention, the antigen contains one or more B cell epitopes. A “B cell epitope” generally refers to the site on an antigen to which a specific antibody molecule binds. The identification of epitopes which are able to elicit an antibody response is readily accomplished using techniques well known in the art. See, eg., Geysen et al., 1984, Proc. Natl. Acad. Sci. USA, 81:3998-4002 (general method of rapidly synthesising peptides to determine the location of immunogenic epitopes in a given antigen); U.S. Pat. No. 4,708,871 (procedures for identifying and chemically synthesising epitopes of antigens); and Geysen et al., 1986, Molecular Immunology, 23:709-715 (technique for identifying peptides with high affinity for a given antibody).

The terms “TH1-associated antigen(s)” or “TH2-associated antigen(s)” as used herein refers to antigens as defined above, but these antigens are specifically associated with the production of a predominantly TH1 or TH2 specific immune response. For example, the major allergen of house dust mite, der P1, produces a predominantly TH2 response in an individual, while P6 outer membrane proteins of Haemophilus influenzae produces a predominantly TH1 response in an individual. Determination of whether an antigen produces a predominantly TH1 or TH2 response in an individual is well within the skill of a person in the art. Following is a list of antigens that may be useful in the present invention.

Useful antigens for treating allergy in the methods of the invention. Antigens of interest include those of animals, including the mite (eg., Dermatophagoides pteronyssinus, Dermatophagoides farinae, Blomia tropicalis), such as the allergens der p1 (Scobie et al., 1994, Biochem. Soc. Trans. 22: 448S; Yssel et al., 1992, J. Immunol. 148: 738-745), der p2 (Chua et al., 1996, Clin. Exp. Allergy, 26: 829-837), der p3 (Smith & Thomas, 1996, Clin. Exp. Allergy, 26: 571-579), der p5, der p V (Lin et al., 1994, J. Allergy Clin. Immunol. 94: 989-996), der p6 (Bennett & Thomas, 1996, Clin. Exp. Allergy, 26: 1150-1154), der p 7 (Shen et al., 1995, Clin. Exp. Allergy, 25: 416-422), der f2 (Yuuki et al., 1997, Int. Arch. Allergy Immunol. 112: 44-48), der f3 (Nishiyama et al. (1995) FEBS Lett. 377: 62-66), der f7 (Shen et al. (1995) Clin. Exp. Allergy 25: 1000-1006); Mag 3 (Fujikawa et al. (1996) Mol. Immunol. 33: 311-319). Also of interest as antigens are the house dust mite allergens Tyr p2 (Eriksson et al. (1998) Eur. J. Biochem. 251: 443-447), Lep d1 (Schmidt et al. (1995) FEBS Lett. 370: 11-14), and glutathione S-transferase (O'Neill et al. (1995) Immunol Lett. 48: 103-107); the 25,589 Da, 219 amino acid polypeptide with homology with glutathione S-transferases (O'Neill et al. (1994) Biochim. Biophys. Acta. 1219: 521-528); Blo t 5 (Arruda et al. (1995) Int. Arch. Allergy Immunol. 107: 456-457); bee venom phospholipase A2 (Carballido et al. (1994) J. Allergy Clin. Immunol. 93: 758-767; Jutel et al. (1995) J. Immunol. 154: 4187-4194); bovine dermal/dander antigens BDA 11 (Rautiainen et al. (1995) J. Invest. Dermatol. 105: 660-663) and BDA20 (Mantyjarvi et al. (1996) J. Allergy Clin. Immunol. 97: 1297-1303); the major horse allergen Equ cl (Gregoire et al. (1996) J. Biol. Chem. 271: 32951-32959); Jumper ant M. pilosula allergen Myr p I and its homologous allergenic polypeptides Myr p2 (Donovan et al. (1996) Biochem. Mol. Biol. Int. 39: 877-885); 1-13, 14, 16 kD allergens of the mite Blomia tropicalis (Caraballo et al. (1996) J. Allergy Clin. Immunol. 98: 573-579); the cockroach allergens Bla g Bd90K (Helm et al. (1996) J. Allergy Clin. Immunol. 98: 172-80) and Bla g 2 (Arruda et al. (1995) J. Biol. Chem. 270: 19563-19568); the cockroach Cr-PI allergens (Wu et al. (1996) J. Biol. Chem. 271: 17937-17943); fire ant venom allergen, Sol i 2 (Schmidt et al. (1996) J. Allergy Clin. Immunol. 98: 82-88); the insect Chironomus thummi major allergen Chi t 1-9 (Kipp et al. (1996) Int. Arch. Allergy Immunol. 110: 348-353); dog allergen Can f 1 or cat allergen Fel d 1 (Ingram et al. (1995) J. Allergy Clin. Immunol. 96: 449-456); albumin, derived, for example, from horse, dog or cat (Goubran Botros et al. (1996) Immunology 88: 340-347); deer allergens with the molecular mass of 22 kD, 25 kD or 60 kD (Spitzauer et al. (1997) Clin. Exp. Allergy 27: 196-200); and the 20 kd major allergen of cow (Ylonen et al. (1994) J. Allergy Clin. Immunol. 93: 851-858).

Pollen and grass allergens are also useful in antigens. Such allergens include, for example, Hor v9 (Astwood and Hill (1996) Gene 182: 53-62, Lig vl (Batanero et al. (1996) Clin. Exp. Allergy 26: 1401-1410); Lol p 1 (Muller et al. (1996) Int. Arch. Allergy Immunol. 109: 352-355), Lol p II (Tamborini et al. (1995) Mol. Immunol. 32: 505-513), Lol PVA, Lol pVB (Ong et al. (1995) Mol. Immunol. 32: 295-302), Lol p 9 (Blaher et al. (1996) J. Allergy Clin. Immunol. 98: 124-132); Par J I (Costa et al. (1994) FEBS Lett. 341: 182-186; Sallusto et al. (1996) J. Allergy Clin. Immunol. 97: 627-637), Par j 2.0101 (Duro et al. (1996) FEBS Lett. 399: 295-298); Bet v1 (Faber et al. (1996) J. Biol. Chem. 271: 19243-19250), Bet v2 (Rihs et al. (1994) Int. Arch. Allergy Immunol. 105: 190-194); Dac g3 (Guerin-Marchand et al. (1996) Mol. Immunol. 33: 797-806); Phl p 1 (Petersen et al. (1995) J. Allergy Clin. Immunol. 95: 987-994), Phl p 5 (Muller et al. (1996) Int. Arch. Allergy Immunol. 109: 352-355), Phl p 6 (Petersen et al. (1995) Int. Arch. Allergy Immunol. 108: 55-59); Cry j I (Sone et al. (1994) Biochem. Biophys. Res. Commun. 199: 619-625), Cry j II (Namba et al. (1994) FEBS Lett. 353: 124-128); Cor a 1 (Schenk etal. (1994) Eur. J. Biochem. 224: 717-722); cyn dl (Smith et al. (1996) J. Allergy Clin. Immunol. 98: 331-343), cyn d7 (Suphiogluet al. (1997) FEBS Lett. 402: 167-172); Pha a 1 and isoforms of Pha a 5 (Suphioglu and Singh (1995) Clin. Exp. Allergy 25: 853-865); Cha o 1 (Suzuki et al. (1996) Mol. Immunol. 33: 451-460); profilin derived, e.g, from timothy grass or birch pollen (Valenta et al. (1994) Biochem. Biophys. Res. Commun. 199: 106-118); P0149 (Wu et al. (1996) Plant Mol. Biol. 32: 1037-1042); Ory sl (Xu et al. (1995) Gene 164: 255-259); and Amb a V and Amb t 5 (Kim et al. (1996) Mol. Immunol. 33: 873-880; Zhu et al. (1995) J. Immunol. 155: 5064-5073).

Fungal allergens include, but are not limited to, the allergen, Cla h III, of Cladosporium herbarum (Zhang et al. (1995) J. Immunol. 154: 710-717); the allergen Psi c 2, a fungal cyclophilin, from the basidiomycete Psilocybe cubensis (Homer et al. (1995) Int. Arch. Allergy Immunol. 107: 298-300); hsp 70 cloned from a cDNA library of Cladosporium herbarum (Zhang et al. (1996) Clin Exp Allergy 26: 88-95); the 68 kD allergen of Penicillium notatum (Shen et al. (1995) Clin. Exp. Allergy 26: 350-356); aldehyde dehydrogenase (ALDH) (Achatz et al. (1995) Mol Immunol. 32: 213-227); enolase (Achatz et al. (1995) Mol. Immunol. 32: 213-227); YCP4 (Id.); acidic ribosomal protein P2 (Id.).

In one embodiment, the antigen is a recombinant antigen expressed in plants or foodstuff. For example, mite antigen Der P1 cloned into banana or yoghurt bacteria.

Screening of optimised antigens can be done in animal models which are known to those of skill in the art. Examples of suitable models for various conditions include collagen induced arthritis, the NFS/sld mouse model of human Sjogren's syndrome; a 120 kD organ-specific autoantigen recently identified as an analog of human cytoskeletal protein (α-fodrin (Haneji et al., 1997, Science, 276: 604), the New Zealand Black/White F1 hybrid mouse model of human SLE, NOD mice, a mouse model of human diabetes mellitus, fas/fas ligand mutant mice, which spontaneously develop autoimmune and lymphoproliferative disorders (Watanabe-Fukunaga et al., 1992, Nature, 356: 314), and experimental autoimmune encephalomyelitis. (EA), in which myelin basic protein induces a disease that resembles human multiple sclerosis.

Once an individual afflicted with a TH1 or TH2-associated disease has been diagnosed and a useful TH1 or TH2 antigen, or combination of antigens, has been identified then an “effective amount” of the antigen(s) is/are administered in immunotherapeutic form to the individual.

The terms “administration,” administering,” and “administered” are used herein interchangeably. The antigen may be administered orally including sublingual, topically, or parenterally in dosage unit formulations containing conventional non-toxic pharmaceutically acceptable carriers, adjuvants, and vehicles. The term parenteral as used herein includes subcutaneous injections, aerosol, intravenous, intramuscular, intrathecal, intracranial, injection or infusion techniques or rectal or vaginally. Preferably, the antigen is administered as a composition containing the antigen and a pharmaceutically acceptable carrier or diluent compatible with the antigen. In preparing such composition, any conventional pharmaceutically acceptable carrier can be utilised.

The carrier material can be organic or inorganic inert carrier material suitable for oral administration. Suitable carriers include water, gelatin, gum arabic, lactose, starch, magnesium stearate, talc, vegetable oils, polyalkylene-glycols, petroleum jelly and the like. Furthermore, the pharmaceutically active preparations may contain other pharmaceutically active agents. Additionally, additives such as flavouring agents, preservatives, stabilizers, emulsifying agents, buffers and the like may be added in accordance with accepted practices of pharmaceutical compounding.

When the antigen is administered orally, it is generally administered at regular intervals, conveniently at meal times or once daily. It has been established that the antigen is effective in doses which show no or only mild side effects when given orally or when given topically. Therefore, oral or topical administration of the antigen is generally preferred.

The antigen preparations can be made up in any conventional form including: (a) solid form for oral, rectal or vaginal administration such as tablets, capsules (e.g. hard or soft gelatine capsules), pills, sachets, powders, granules, and the like; and (b) preparations for topical administrations such as solutions, suspensions, ointments, creams, gels, micronised powders, sprays, aerosols and the like; (c) liquid formulations for intravenous administrated may also be prepared. Pharmaceutical preparations may be sterilised and/or may contain preservatives, stabilisers, wetting agents, emulsifiers, salts for varying the osmotic pressure and/or buffers.

For topical administration to the skin or mucous membrane the aforementioned antigen preparation is preferably prepared as an ointment, tincture, cream, gel, solution, lotion, spray; aerosol and dry powder for inhalation, suspension and the like. In fact, any conventional antigen preparation can be utilised in this invention. Among the preferred methods of applying the antigen preparation containing the antigen(s) of this invention is in the form of an ointment, gel, cream, lotion, spray; aerosol or dry powder for inhalation. A pharmaceutical preparation for topical administration to the skin can be prepared by mixing the aforementioned antigen preparation with non-toxic, therapeutically inert, solid or liquid carriers customarily used in such preparation. These preparations generally contain 0.01 to 5.0 percent by weight, preferably 0.1 to 1.0 percent by weight, of the antigen, based on the total weight of the antigen preparation.

In preparing the topical preparations described above, additives such as preservatives, thickeners, perfumes and the like conventional in the art of pharmaceutical compounding of topical preparation can be used. In addition, conventional antioxidants or mixtures of conventional antioxidants can be incorporated into the topical preparations containing the afore-mentioned active agent. Among the conventional antioxidants which can be utilized in these preparations are included N-methyl-α-tocopherolamine, tocopherols, butylated hydroxyanisole, butylated hydroxytoluene, ethoxyquin and the like. Cream-base pharmaceutical formulations containing the antigen preparation, used in accordance with this invention, are composed of aqueous emulsions containing a fatty acid alcohol, semi-solid petroleum hydrocarbon, ethylene glycol and an emulsifying agent.

Ointment formulations containing the antigen preparation in accordance with this invention comprise admixtures of a semi-solid petroleum hydrocarbon with a solvent dispersion of the antigen. Cream compositions containing the antigen preparation for use in this invention preferably comprise emulsions formed from a water phase of a humectant, a viscosity stabiliser and water, an oil phase of a fatty acid alcohol, a semi-solid petroleum hydrocarbon and an emulsifying agent and a phase containing the antigen preparation dispersed in an aqueous stabiliser-buffer solution. Stabilisers may be added to the topical preparation. Any conventional stabiliser can be utilised in accordance with this invention. In the oil phase, fatty acid alcohol components function as a stabiliser. These fatty acid alcohol components function as a stabiliser. These fatty acid alcohol components are derived from the reduction of a long-chain saturated fatty acid containing at least 14 carbon atoms.

Formulations for aerosols are described in Drugs and Pharmaceutical Sciences, Marcel Dekker, New York, 72: 547-574 (1996). Furthermore, the antigen preparation can be delivered by dry powder inhalation. Such formulations and devices are described in Pharmaceutical Technology, June 1997, pp.117-125.

Depending upon the mode or type of administration, the type of disease and the antigen used, the treatment regime will vary. However, typically an individual is monitored daily, weekly or monthly, depending on the above factors, and the status of their specific immune response is determined. Administration of the antigen(s) continues until the specific immune response is down regulated. After which the individual is then administered the same antigen(s) in an immunogenic form.

An “immunogenic form” of an antigen is a form of or formulation comprising the antigen, which renders the antigen immunogenic. Such forms include, but are not limited to, antigen alone, antigen in conjunction with one or more TH1 or TH2-associated adjuvants, antigen in association with or conjugated to a moiety, such as a hapten.

The term “an antigen administered in immunogenic form” as used herein also refers to the type of administration and route of administration relative to the type or route of administration used for the immunotherapeutic form of antigen. For example, in one embodiment of the present invention the immunogenic form of the antigen will be administered subcutaneously, while the same antigen used to desensitise an individual (immunotherapeutic form) might be administered sublingually.

In one preferred embodiment, the immunogenic form of the antigen comprises antigen together with an appropriate TH1 or TH2 adjuvant ie one that is normally associated with inducing the type of TH-response which is the target of the immunotherapy. Generally, the term “adjuvant” refers to a substance which, when added to an immunogenic agent, non-specifically enhances or potentiates an immune response to the agent in the recipient host upon exposure to the mixture. However, as used herein the term “adjuvant” refers to either “TH1 adjuvant” or “TH2 adjuvant. Typically, TH1 adjuvants, or immunostimulants, induce an increase of TH1 cytokines (eg IFN γ) production. TH2 adjuvants induce an increase of TH2 cytokines (eg IL-4) production.

Preferred adjuvants for use in eliciting a predominantly TH1-type response include, for example, complete Freund's adjuvant, a combination of monophosphoryl lipid A, preferably 3-de-O-acylated monophosphoryl lipid A (3D-MPL), together with an aluminum salt. MPL adjuvants are available from Ribi ImmunoChem Research Inc. (Hamilton, Mont.; see U.S. Pat. Nos. 4,436,727; 4,877,611; 4,866,034 and 4,912,094). CpG-containing oligonucleotides (in which the CpG dinucleotide is unmethylated) also induce a predominantly TH1 response. Such oligonucleotides are well known and are described, for example, in WO 96/02555, Wo 99/33488 and U.S. Pat. Nos. 6,008,200 and 5,856,462. Immunostimulatory DNA sequences are also described, for example, by Sato et al., Science 273:352, 1996 and immunostimulatory nucleotide sequence (ISS) as disclosed in US Pat. No. 6,514,948. Another preferred TH1 adjuvant is a saponin, preferably QS21 (Aquila, United States), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210. By virtue of its ability to induce an exclusive TH1 immune response, the use of the L. braziliensis ribosomal antigen (LbeIF4A), and variants thereof, as an adjuvant is also anticipated.

Other preferred TH1 adjuvants include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Corixa, Hamilton, Mont.), RC-529 (Corixa, Hamilton, Mont.) and other aminoalkyl glucosaminide 4-phosphates (AGPs), such as those described in U.S. Pat. Nos. 6,113,918 and 6,355,257, the disclosures of which are incorporated herein by reference in their entireties.

Preferred adjuvants for use in eliciting a predominantly TH2-type response include, for example, phosphopolymer (Guy et al. 1998, Vaccine 16:850-856.) and alum (eg., aluminium hydroxide, aluminium phosphate).

Other useful adjuvants include cholera toxin, procholeragenoid, cholera toxin B subunit and fungal polysaccharides including, but not limited to, schizophyllan, muramyl dipeptide, muramyl dipeptide derivatives, phorbol esters, microspheres, non-Helicobacter pylori bacterial lysates, labile toxin of Escherichia coli, block polymers, saponins, and ISCOMS. For additional adjuvants, those of ordinary skill in the art may also refer to, for example, Azuma, 1992, Vaccine, vol. 10, 1000 (1992); Pockley & Montgomery, 1991, Immunology, vol. 73, 19-23; Adam & Lederer “Muramyl peptides as Immunomodulators” ISI Atlas of Science 205 (1988); Clements et al. 1988, Vaccine, vol. 6, 269; Ben Ahmeida et al., 1993, Vaccine, vol. 11, 1302; and Gupta, et al., 1993, Vaccine, vol. 11, 290-308.

In one embodiment the antigen(s) and or adjuvant(s) are incorporated into a single immunomodifying agent. As used herein the term “immunomodifying agent” refers to a formulation comprising at least one TH1 or TH2 antigen. In one embodiment, the immunomodifying agent further comprises at least one TH1 and/or TH2 adjuvant. The use of TH1 and/or TH2 adjuvant will depend upon whether the disease or condition being treated is a TH1- or TH2-associated disease.

The amount of immunomodifying agent administered to an individual is described as an “effective amount”. As used herein, the term “effective amount” means an amount of one or more antigens of the present invention in immunogenic form, which is/are capable of producing a therapeutic response. For example, in the present invention this would be an amelioration of the clinical symptoms of TH1 or TH2-associated diseases. The “effective amount” of the immunomodifying agent would effect a reversal of the TH1 or TH2 specific immune response. The reversal would be an effective change in response from, for example, a predominantly TH1 type response to a predominantly TH2 type response or vice versa. The reversal may be brought about by selective enhancement of one TH cell type over that of the other phenotype or the selective down-regulation of one TH cell type over that of the other TH cell type.

The specific “effective amount” will, obviously, vary with such factors as the particular condition being treated, the physical condition of the patient, the type of individual being treated, the duration of the treatment, the nature of concurrent therapy (if any), and the specific formulations employed and the structure of the immunomodifying agent.

As for the antigen preparation described previously, the immunomodifying agent may be used in combination with suitable “pharmaceutical carriers” such as pharmaceutically acceptable solvents, suspending agents or vehicles for delivering the immunomodifying agent of the present invention to the individual being treated. The carrier may be liquid or solid and is selected with the planned manner of administration in mind.

In one embodiment, the antigen(s), adjuvant(s) and/or immunomodifying agent can be provided in the form of a kit comprising TH1 or TH2 antigen and/or TH1 or TH2 adjuvant and any additional medicaments, as well as a device for delivery of the antigen or adjuvant to an individuals tissue and reagents for determining the biological effect of the antigen or adjuvant on a treated individual. Throughout the specification, unless the context requires otherwise, the word “comprise” or variations such as “comprises” or “comprising”, will be understood to imply the inclusion of a stated integer or group of integers but not the exclusion of any other integer or group of integers.

The invention will now be further described by way of reference only to the following non-limiting examples. It should be understood, however, that the examples following are illustrative only, and should not be taken in any way as a restriction on the generality of the invention described above. In particular, while the invention is described in detail in relation to the use of specific TH1 and TH2 antigens and adjuvants, it will be clearly understood that the findings herein are not limited to these antigens or adjuvants.

### EXAMPLE 1

Selective Tolerisation of TH2 Immunity

Specific pathogen free C57BL/6J and BALB/c mice were purchased from the Animal Resource Centre (Murdoch University, Western Australia) and housed under barrier conditions at the Telethon Institute for Child Health Research. The animals were maintained on temperature and light controlled environment and housed on low-dust bedding. Animals were fed a diet of acidified water and autoclaved OVA-free food pellets. Advanced pregnant females were monitored daily at 9 am and 5 pm for the date of delivery. Birth day was designated day 0. Neonatal animals were defined as 24 h old. Adults were used at 6-8 weeks of age. All animal experimentation was approved by the Institute's Animal Ethics and Experimentation Committee, which complies with the conditions set down by the National Health and Medical Research Council of Australia.

Adult mice were fed 3 ×1 mg OVA (grade V; Sigma, MO, USA) was dissolved in PBS at a concentration of 100 mg/ml or PBS on 3 consecutive days by gastric intubation. 4 weeks later they were challenged ip with 100 μg OVA in Aluminium Hydroxide adjuvant 4 mg 11 days later draining lymph node cells were stimulated in vitro with 1 mg/ml OVA and culture supernatants assayed for IFN γ and IL-5 by capture ELISA as per manufacturer's instructions (all from Pharmingen; San Diego, USA). The concentrations of IFN γ and IL-5 in the culture supernatant were interpolated from the linear portion of the standard curve with known amounts of recombinant IFN γ and IL-5 using Assayzap universal calculator software. The results are expressed in pg/ml and sensitivity of ELISA assays were 15 pg/ml for IFN γ and 40 pg/ml for IL-5.

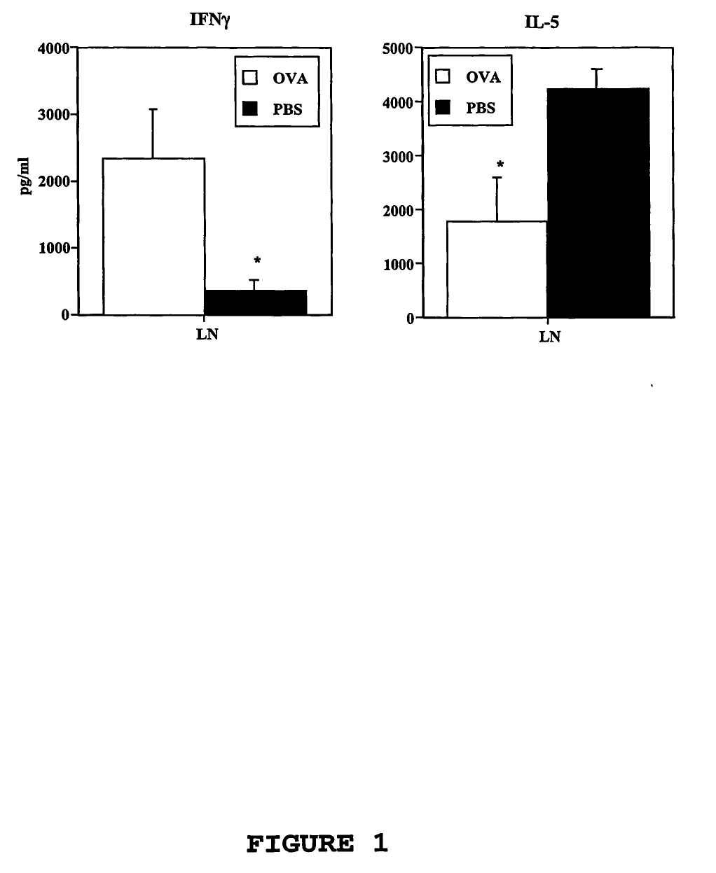

FIG. 1 shows the results expressed as mean ± SEM from groups of 6 mice and compared using an unpaired Student's t test. The results were analysed using the Instat software program, version 2 (Graphpad software, San Diego, USA) for MacIntosh computers. Differences were considered as significant when p value <0.05. The results indicate selective tolerisation of TH2 immunity as shown by decreased in vitro production of the TH2 cytokine IL-5 in OVA-fed mice post challenge with OVA in Aluminium Hydroxide, and accompanying increased production of the TH1 cytokine IFN-γ.

### EXAMPLE 2

Selective Tolerisation of TH1 Immunity

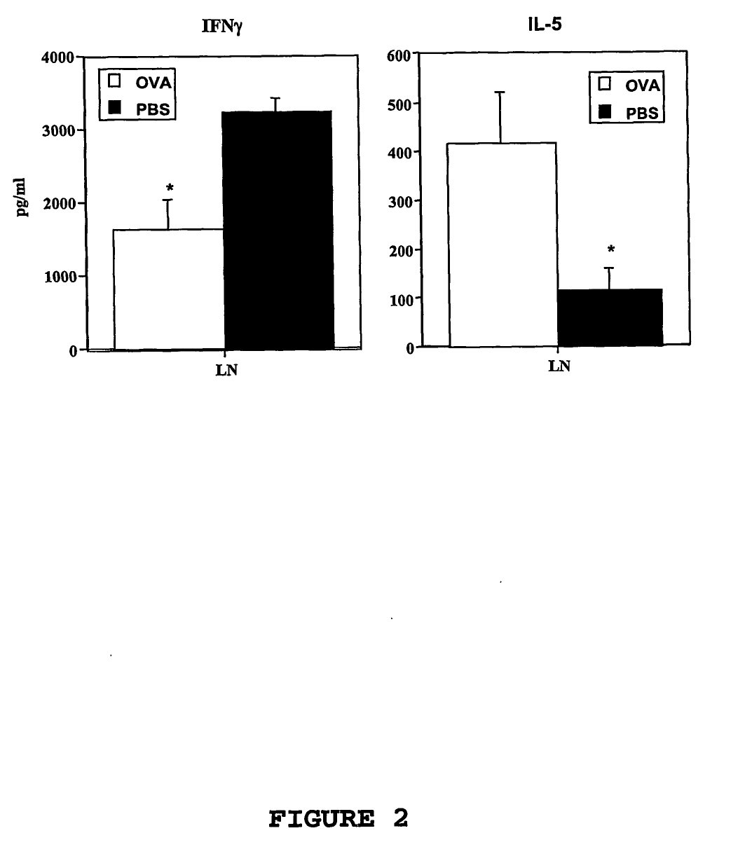

As in Example 1 above, adult mice were fed 3×1 mg OVA or PBS on 3 consecutive days. However, after 4 weeks they were challenged ip with 100 μg OVA in Complete Freund's adjuvant. Again, 11 days later draining lymph node cells were stimulated in vitro with 1 mg/ml OVA and culture supernatants assayed for cytokines as described in Example 1. FIG. 2 shows the selective tolerisation of TH1 immunity as demonstrated by decreased in vitro production of the TH1 cytokine IFN-γ in OVA-fed mice post challenge with OVA in Complete Freunds Adjuvant, and accompanying increased production of the TH2 cytokine IL-5.

### EXAMPLE 3

Non-Selective Tolerisation of Overall Ovaspecific TH-Cell Immunity

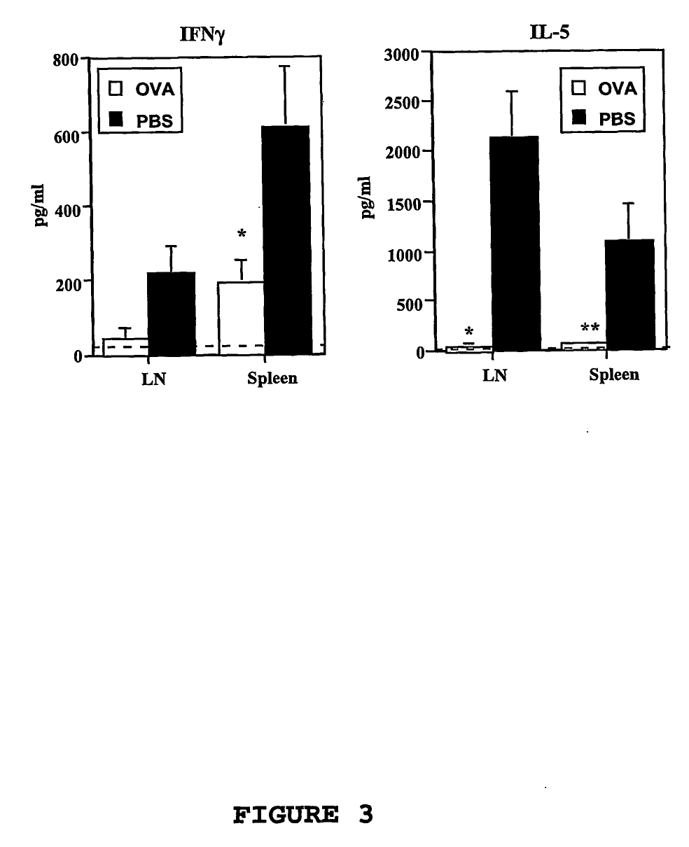

As in Examples 1 and 2, adult mice were fed 3×1 mg OVA or PBS on 3 consecutive days. However, 4 weeks later they were challenged ip with 100 μg soluble OVA in PBS. Again, 11 days later draining lymph node and spleen cells were stimulated in vitro with 1 mg/ml OVA and culture supernatants assayed for cytokines as described above. FIG. 3 shows the non-selective tolerisation of overall OVA specific TH-cell immunity as demonstrated by parallel reductions in in vitro production of both IL-5 and IFN-γ in animals after challenge with soluble OVA without adjuvant.

### EXAMPLE 4

Desensitisation of Ova-Sensitised Mice

Three groups of mice were sensitised to OVA by ip immunisation with 1 μg OVA in the TH2-selective adjuvant aluminium hydroxide (AH) on day 0. One group (Group C) were then given s.c. injections of 25 μg OVA repeatedly on days 7, 9, 14, 16, 21, 23, 28, 30 and 31, aimed at “desensitisation” of their TH2-dependent IgE responses immunotherapy protocol]. A second group instead received repeated PBS injections (Group B), and a third group received no further treatment up until day 32 (Group A). On day 32, all 3 groups were challenged ip with a further dose of OVA in AH. All animals were then bled for IgE anti-OVA assays on days 31 and 51.

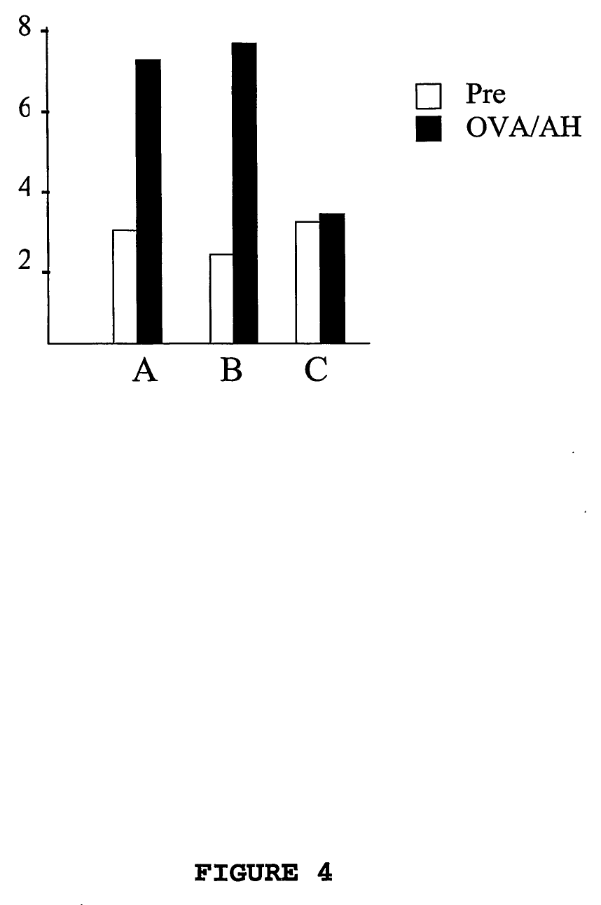

It can be seen in FIG. 4 that group C was desensitised (tolerised), as shown by their inability to mount a secondary IgE response to the OVA/AH challenge. In contrast, Groups A and B displayed strong secondary IgE responses, as shown by an approximately x3 increase in IgE antibody titres on day 51.

These data provide proof-of-principle that challenge of animals “allergic” to OVA after a course of desensitising injections of the allergen [immunotherapy protocol], with the same allergen in a TH2-skewing adjuvant, will result in desensitisation/tolerisation of TH2-dependent IgE responses. The “immunotherapy protocol” mimics the type of treatment currently given to allergic humans to cure their allergy. We hypothesize that addition of the allergen/AH challenge at the end of the “immunotherapy protocol” will function like a “booster injection” to increase the efficiency of down regulation of the IgE response by selectively directing the tolerance process towards the TH2 arm of the immune response.

### EXAMPLE 5

Sublingually Treatment

As described in Example 4, mice were sensitised with ip administration of lg of OVA (antigen) in 4mg of alum (TH2-selective adjuvant) on day 0. This effectively established mice that were “allergic” to OVA.

Mice were then given 5 daily sublingual doses of OVA on the weeks starting on day 7, 14, 21 and 28 (a total of 20 doses). Each dose being 100 μg in 10 μl PBS. Control mice received the same volume of PBS only. This treatment step represented the “immunotherapeutic” step typically described as immunotherapy, wherein animals are given doses of the allergy-producing antigen and expected to progressively lose their sensitivity to it.

On day 37 the mice were then split into three groups:
1). “Control” group. These mice received the “typical immunotherapy”, where the treatment only consisted of the immunotherapeutic administration of sublingual OVA.
2). These mice received the “novel” treatment regimen of a sublingual dose of immunotherapeutic (ending day 32) and followed on day 37 by the immunogenic administration. The immunogenic form was ip injection with 100 μg of OVA in PBS (i.e. IgE soluble challenged group); and
3). These mice received the “novel” treatment regimen of a sublingual dose of immunotherapeutic (ending day 32) and followed on day 37 by the immunogenic administration. The immunogenic form was ip injection with 100 μg of OVA in 4mg of alum (TH2 adjuvant) (i.e. IgE alum challenge group).

The mice received further boosts of 100 μg of OVA ip in PBS on days 75, 158 and 206.

The anti-OVA IgE antibody levels in mouse sera were then titrated by the PCA test in rats (Ovary & Kojima, 1975, International Arch. Allergy & Appl. Immunol., 48:16) using the 24-h latent period for skin sensitisation. Briefly, serum samples were serially diluted in PBS and injected intradermally in 5 μl aliquots into the dorsal skin of male WAG rates. The PCA reaction was evoked 24 hours later by intravenous challenge with 4 mg/ml OVA in PBS containing of 1% Evans' Blue dye. Fifteen minutes later, the skin was examined for development of blue lesions. The reciprocal of the highest dilution of the serum giving a blue lesion of 5 mm diameter was taken as the PCA titre. Serum collected from mice that were given multiple injections of OVA in alum was used as positive controls. Serum taken from mice that were given multiple injections of PBS was used as negative controls.

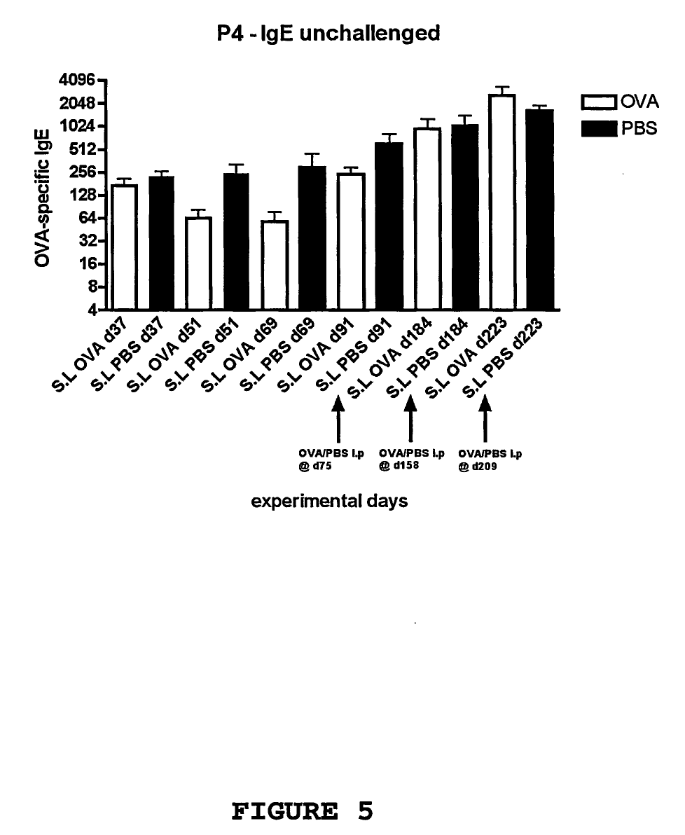

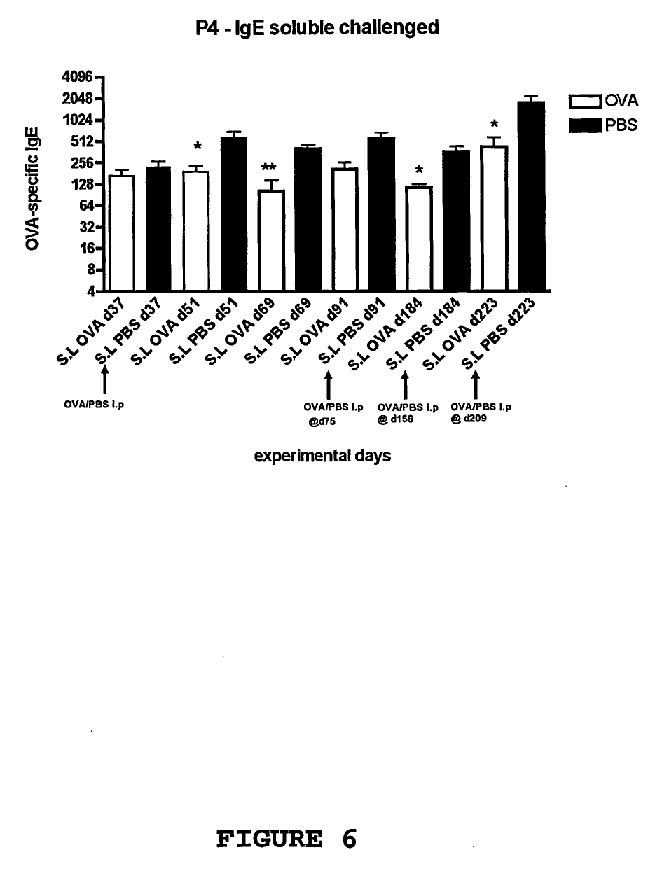

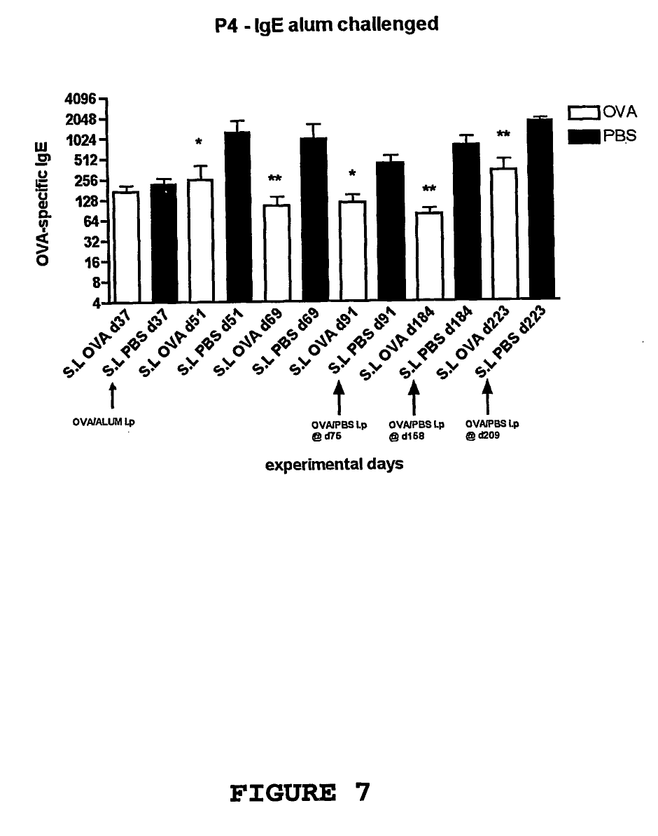

FIGS. 5 to 7 show the PCA titres of serum taken at the days indicated. “S.L OVA” are the mice that received the OVA sublingually and “S.L. PBS” are the mice that received PBS sublingually (also represented by the black and white bars).

The results show that:
1. The significant differences between the S.L.-PBS and the S.L. OVA were only found in the mice challenged on day 37 with OVA (in PBS or alum).
2. The difference remained significant over an extended time course even though the ultimate titres of the controls (S.L. PBS) of unchallenged group and challenged groups were the same.
3. The IgE titres of the mice given S.L. OVA and then challenged were lower than the SL groups that were not challenged.
4. The titres of S.L. treated mice that were challenged with OVA and alum were the lowest of any group.

These data demonstrate the principle that parenteral treatment with antigen/adjuvant following sublingual immunotherapy (“desensitisation”) enhances the efficiency of the desensitisation process. In other words, treatment with antigen/adjuvant following immunotherapy “boosts” desensitisation process.

The claims defining the invention are as follows:

1. A method of altering a specific immune response to an antigen in an individual sensitized to the antigen comprising:
i). administering to the individual an effective amount of the antigen in immunotherapeutic form, wherein said immune response is down regulated; and
ii). subsequently administering to the individual an effective amount of an immunomodifying agent comprising the antigen in immunogenic form.

2. A method according to claim 1, wherein the immunomodifying agent further comprises either a TH1 or TH2 adjuvant, wherein the adjuvant normally induces the type of TH-response which is the target of the immunotherapy.

3. A method according to claim 1 or claim 2, wherein the immunotherapy is targeted at the specific immune response.

4. A method according to any one of claims 1 to 3, wherein the effective amount in step i) is one or more doses of said antigen in immunotherapeutic form.

5. A method according to any one of claims 1 to 4, wherein said antigen in immunotherapeutic form further comprises agents designed to modulate the specific immune responses.

6. A method according to any one of claims 1 to 5, wherein the alteration to the specific immune response is attenuation of the TH-response component, which is associated with expression of the disease being treated.

7. A method according to any one of claims 1 to 5, wherein the alteration to the specific immune response is conversion of the TH1 component of the response to a TH2 component or conversion of the TH2 component to a TH1 component.

8. A method according to any one of claims 1 to 5, wherein the alteration to the specific immune response is reversing the ratio between the TH1 and TH2 components of the response.

9. A method according to claim 8, wherein the immune response in an untreated individual comprised high level production of TH1 cytokines and low level production of TH2 cytokines is reversed following treatment.

10. A method according to claim 8, wherein the immune response in an untreated individual comprised high level production of TH2 cytokines and low level production of TH1 cytokines is reversed following treatment.

11. A method of treating a TH1-associated disease comprising:
i). administering to an individual in need thereof an effective amount of an antigen in immunotherapeutic form; and
ii). subsequently administering to the individual an effective amount of an immunomodifying agent comprising said antigen in immunogenic form, wherein the antigen specific TH1 response in the individual is reduced relative to the specific TH1 response before administration of said immunomodifying agent.

12. A method according to claim 11, wherein the immunomodifying agent further comprises a TH1 adjuvant.

13. A method of treating a TH2-associated disease comprising:
i). administering to an individual in need thereof an effective amount of an antigen in immunotherapeutic form; and
ii). subsequently administering to the individual an effective amount of an immunomodifying agent comprising said antigen in immunogenic form, wherein the antigen specific TH2 response in the individual is reduced relative to the specific TH2 response before administration of said immunomodifying agent.

14. A method according to claim 13, wherein the immunomodifying agent further comprises a TH2 adjuvant.

15. A method of treating a disease associated with a mixed TH1 and TH2 immune response comprising:
i). administering to an individual in need thereof an effective amount of an antigen in immunotherapeutic form; and
ii). subsequently administering to the individual an effective amount of an immunomodifying agent comprising said antigen in immunogenic form which boosts both TH1 and TH2 immunity, wherein ensuing specific TH1 and TH2 responses in the individual are reduced relative to the specific TH1 and TH2 responses before administration of said immunomodifying agent.

16. A method according to claim 15, wherein the immunotherapeutic form in step i) is sublingual administration of antigen.

17. A method according to claim 15 or claim 16, wherein the immunomodifying agent in step ii) is administered parenterally.

19. A method according to any one of claims 15 to 18, wherein the immunomodifying agent further comprises either an adjuvant which boosts both TH1 and TH2 immunity or a mixture of TH1 and TH2 adjuvants, wherein ensuing specific TH1 and TH2 responses in the individual are reduced relative to the specific TH1 and TH2 responses before administration of said immunomodifying agent.

20. A method according to claim 1, wherein the immunotherapy is administration to an individual in need thereof an effective amount of one or more antigen(s) in immunotherapeutic form, wherein the antigens are associated with expression of pathogenic TH2 immunity.

21. A method according to claim 21, wherein the individual suffers from a TH1-associated disease and the antigen in immunotherapeutic form is predominately a TH1-specific antigen.

22. A method of treating a disease comprising:
i). administering to an individual in need thereof an effective amount of an antigen in immunotherapeutic form, wherein the immune response to said disease is down regulated; and
ii). subsequently administering to the individual an effective amount of an immunomodifying agent comprising said antigen in immunomodifying form.

23. A method according to claim 22, wherein the immunomodifying agent further comprises either a TH1 or TH2 adjuvant, wherein the adjuvant normally induces the type of TH-response which is the target of the immunotherapeutic form of the antigen.

24. A method according to claim 22, wherein the disease is a TH1-associated disease selected from the group consisting of rheumatoid arthritis, multiple sclerosis, thyroiditis, Crohn's disease, systemic lupus erythematosus, experimental autoimmune uveoretinitis, experimental autoimmune encephalitis, insulin dependent diabetes mellitus, contact dermatitis and chronic inflammatory disorders.

25. A method according to claim 22, wherein the disease is a TH2-associated disease selected from the group consisting of allergic atopic disorders, allergic asthma, atopic dermatitis, hyper-IgE syndrome, Omenn's syndrome, and allergic rhinitis.

26. A method according to claim 2, wherein the TH2 adjuvant is selected from the group consisting of alum, pertussis toxin, lacto fucopentaose III, and phosphopolymer or combinations thereof.

27. A method according to claim 2, wherein the TH1 adjuvant is selected from the group consisting of complete Freund's adjuvant, monophosphoryl lipid A, 3-de-O-acylated monophosphoryl lipid A (3D-MPL), aluminum salt, CpG-containing oligonucleotides, immunostimulatory DNA sequences, saponin, Montanide ISA 720, SAF, ISCOMS, MF-59, SBAS-3, SBAS-4, Detox, RC-529, aminoalkyl glucosaminide 4-phosphate, and LbeIF4A.

28. A method according to any one of claims 1 to 27, wherein the individual is a mammalian animal.

29. A method according to claim 28, wherein the mammalian animal is a dog, a cat, a livestock animal, a primate or a horse.

30. A method according to claim 28, wherein the mammalian animal is a human.

31. A kit when used for altering TH1 or TH2 response phenotype in an individual in need thereof comprising:
i). one or more TH1 antigen(s); or
ii). one or more TH1 or TH2 adjuvant(s); or
iii).combinations thereof; and
iv). instructions for use.

32. A method of immunotherapy comprising:
i). administration to an individual in need thereof a plurality of antigen shots;
ii). administration to said individual less than five individual shots of said antigen combined with one or more TH1 and/or TH2 adjuvant(s).

33. A method according to claim 32, wherein the individual shots of said antigen combined with TH1 and/or TH2 adjuvant is less than three.

34. A method according to claim 32, wherein the individual shots of said antigen combined with TH1 and/or TH2 adjuvant is one.

35. Use of an immunomodifying agent for the manufacture of a medicament for the treatment a TH1-associated disease or TH2-associated disease, wherein said immunomodifying agent comprises an antigen in immunomodifying form.

36. Use according to claim 35, wherein the immunomodifying agent further comprises at least one adjuvant that is associated with augmenting a T helper-response of the type associated with said disease.

37. Use of immunomodifying agent for the manufacture of a medicament for the treatment of a TH-1 or TH-2 associated disease inflicting an individual susceptible hereto, where said individual previously is treated with an immunotherapeutic form and dose of an antigen having reduced the T-helper immune response associated with said disease in said individual, and wherein the immunomodifying agent comprises at least one adjuvant that is associated with augmenting a T helper-response of the type associated with said disease and a immunogenic form of said antigen.

38. Use according to claim 37, wherein the immunotherapeutic form is targeted at the specific immune response.

39. Use according to claim 37, wherein the alteration to the specific immune response is attenuation of the TH-response component, which is associated with expression of the disease being treated.

40. Use according to claim 37, wherein the alteration to the specific immune response is conversion of the TH1 component of the response to a TH2 component or conversion of the TH2 component to a TH1 component.

41. Use according to claim 37, wherein the alteration to the specific immune response is reversing the ratio between the TH1 and TH2 components of the response.

42. Use according to claim 37, wherein the immune response in an untreated individual comprised high level production of TH1 cytokines and low level production of TH2 cytokines is reversed following treatment.

43. Use according to claim 37, wherein the immune response in an untreated individual comprised high level production of TH2 cytokines and low level production of TH1 cytokines is reversed following treatment.

44. Use according to claim 37, wherein the disease is a TH1-associated disease selected from the group consisting of rheumatoid arthritis, multiple sclerosis, thyroiditis, Crohn's disease, systemic lupus erythematosus, experimental autoimmune uveoretinitis, experimental autoimmune encephalitis, insulin dependent diabetes mellitus, contact dermatitis and chronic inflammatory disorders.

45. Use according to claim 37, wherein the disease is a TH2-associated disease selected from the group consisting of allergic atopic disorders, allergic asthma, atopic dermatitis, hyper-IgE syndrome, Omenn's syndrome, and allergic rhinitis.

46. Use according to claim 37, wherein the TH2 adjuvant is selected from the group consisting of alum, pertussis toxin, lacto fucopentaose III, and phosphopolymer or combinations thereof.

47. Use according to claim 37, wherein the TH1 adjuvant is selected from the group consisting of complete Freund's adjuvant, monophosphoryl lipid A, 3-de-O-acylated monophosphoryl lipid A (3D-MPL), aluminum salt, CpG-containing oligonucleotides, immunostimulatory DNA sequences, saponin, Montanide ISA 720, SAF, ISCOMS, MF-59, SBAS-3, SBAS-4, Detox, RC-529, aminoalkyl glucosaminide 4-phosphate, and LbeIF4A.

48. Use according to any one of claims 35 to 47, wherein the individual is a mammalian animal.

49. Use according to 48, wherein the mammalian animal is a dog, a cat, a livestock animal, a primate or a horse.

50. Use according to 48, wherein the mammalian animal is a human.

51. An immunomodifying agent comprising at least one antigen in immunogenic form and at least one adjuvant, wherein the adjuvant normally induces the type of TH-response associated with the disease caused by said antigen.
